(19) United States
(12) Patent Application Publication
(10) Pub. No.: US 2007/0083140 A1
(43) Pub. Date: Apr. 12, 2007

(54) Bandage insert/inset composite

(76) Inventor: Heinz Lohrer, Liederbach (DE)

Correspondence Address: WARE FRESSOLA VAN DER SLUYS &;ADOLPHSON, LLP, BRADFORD GREEN, BUILDING 5, 755 MAIN STREET, P O BOX 224, MONROE, CT, 06468, US

(21) Appl. No.: 10/576,699

(22) PCT Filed: Oct. 21, 2004
(86) PCT No.: PCT/EP04/11920

§ 371 Date: Apr. 21, 2006

(30) Foreign Application Priority Data
Oct. 21, 2003 (DE) 203 16 109.2

(51) Int. Cl. A61F 13/00

(52) U.S. Cl. 602/60

(57) ABSTRACT

An elastic bandage for holding orthopedic or orthotic elements adjacent an injured or injury-prone limb has an elastic sleeve or tube that fits over the limb. The sleeve or tube has at least one holding element, which is preferably a hook and loop fastener, at a defined location on the outer and/or inner surfaces thereof, for detachably fixing orthopedic or orthotic elements having complementary hook and loop fastening areas. The orthopedic or orthotic elements are corrective, supporting, relieving and/or padding inserts or insets, especially insoles.

The invention relates to an elastic bandage in the form of a sleeve or tube for injured or injury-prone limbs according to the preamble to claim 1.

Bandages of this kind that are designed in the form of a sleeve or tube are usually used for correction, support, and/or stress relief of injured or injury-prone limbs.

In lieu of these or in addition to them, orthopedic and/or orthotic elements are also used for the same or a similar purpose, for example inserts or fitting pieces that serve a bedding, supporting, stress-relieving, and/or corrective purpose. A particularly frequent field of application for these is sport shoe inserts in which the main problem is to securely stabilize them in the shoe to prevent slippage and thus to assure a secure frictional engagement between the foot and shoe.

Before now, it has not been possible—or not to a satisfactory degree—to position such orthopedic or orthotic elements in a quick, trouble-free manner at the desired location on the relevant limb and thus to simultaneously be certain that the desired, precise positioning will be maintained over an extended use.

The object of the invention therefore is to create a device that is able to rectify the above-explained disadvantages by being able to position the medically necessary orthopedic or orthotic element in a quick, trouble-free manner at the desired location on the relevant limb without taking up too much space and simultaneously to assure that the desired individual, precise position is always maintained.

The defining characteristics of the invention created to achieve this object are disclosed in claim 1. Advantageous embodiments of the invention are described in the remaining claims.

The invention is essentially based on the concept of providing an intrinsically known elastic bandage in the form of a sleeve or tube in order to position the desired orthopedic or orthotic element. According to the invention, at particular locations on its outer and/or inner surface, this bandage has at least one holder for detachably fastening orthopedic or orthotic elements such as inserts or fitting pieces that serve a corrective, supporting, stress-relieving, and/or bedding purpose, in particular shoe inserts.

According to a preferred embodiment of the invention, the holder is embodied in the form of a pocket into which the insert or fitting piece can be inserted.

In this case, the pocket provided for containing the insert or fitting piece is suitably embodied as open on at least one end, for example its upper end.

Alternatively, the pocket according to the invention can also be open at two opposite ends to permit easier, quicker insertion and removal of the insert or fitting piece.

It is advantageous if the open end of the pocket is embodied as closable. A preferred pocket closure here can be a hook and loop fastener.

According to a modified embodiment form of the invention, the holder can be embodied in the form of a wing-like flap that is attached to the bandage at one edge and can be fastened to the bandage at its other, open edges, in particular by means of a hook and loop fastener.

In the context of the present invention, the flap is depicted as a flat piece that is square, rectangular, or triangular.

In another embodiment of the invention, the holder can also be embodied so that it constitutes a hook and loop fastener surface for accommodating the insert or fitting piece on the bandage. For example, this fastener surface can be provided at the edges, which can be covered by a corresponding, complementarily embodied flat piece for holding the insert or fitting piece.

In another advantageous embodiment of the invention, the fitting piece is affixed in the desired location of the bandage—whether outside or inside—not indirectly as it were, i.e. by means of a pocket for containing it, a flat piece for covering it, or the like, but is instead affixed directly to the bandage. To this end, according to the invention, the holder is embodied in the form of a hook and loop fastener surface that is provided at the desired location or in a desired surface region of the bandage, to which the fitting piece can be directly—detachably—fastened by means of a complementary hook and loop fastener surface attached to the fitting piece. This affords not only the above-mentioned advantage of attaching the fitting piece directly to the bandage, but also permits the fitting piece to be attached with no trouble to any location on the bandage, provided that the hook and loop region on the bandage extends over a larger area of the bandage, for example over its entire sole region.

In any case, the bandage according to the invention achieves the essential advantages of permitting it, together with the inserts or fitting pieces correspondingly secured to it, to be positioned in a quick, trouble-free manner to the foot or another limb in comparison to an insert or fitting piece attached to a bandage or the like while taking up only a small volume and assuring a precise positioning of the insert or fitting piece.

The invention will be explained in greater detail below in conjunction with the drawings.

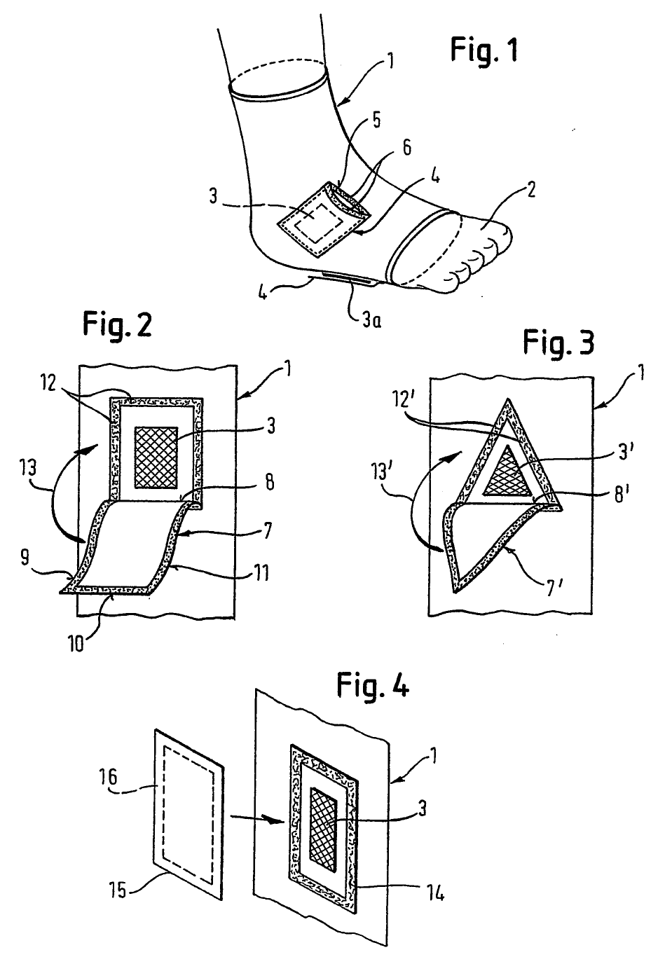

FIG. 1 shows the elastic bandage according to the invention, with an external pocket for holding a support;

FIG. 2 shows a modified embodiment form of this holder in the form of a wing-like flap in a rectangular form;

FIG. 3 shows a triangular form;

FIG. 4 shows another embodiment form of the holder in the form of a hook and loop fastener surface that can be covered by a correspondingly embodied flat piece,

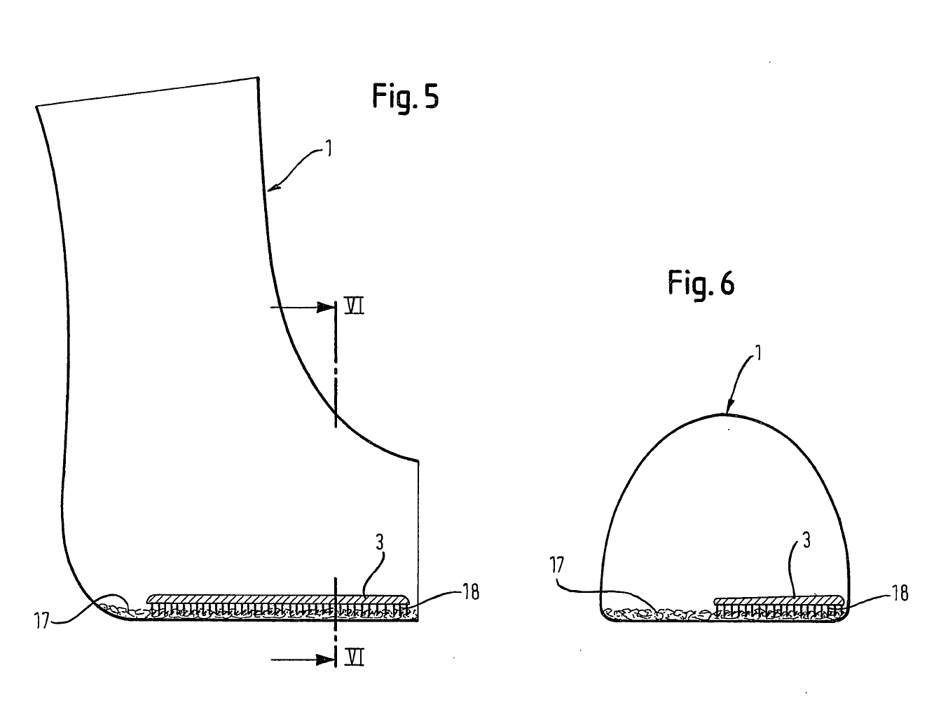

FIG. 5 shows a longitudinal section through another embodiment form of the holder, and

FIG. 6 shows a cross section through this embodiment form along the line VI-VI in FIG. 5.

As is evident from FIG. 1, the elastic bandage 1 depicted is embodied in the form of a sleeve or tube and is provided for use on an injured or injury-prone foot 2 and ankle joint.

A support 3 in the form of an insert, pad, or fitting piece orthopedically prescribed for this purpose should be placed at the level of the ankle to support it without the danger of slippage, i.e. attached elastically. To this end, the bandage 1 has a holder on the outside in the form of a side pocket 4 into which the support 3 can be inserted.

A pocket 4 of this kind is also provided on the sole side of the bandage 1, into which an insert 3a can be detachably inserted and thus precisely positioned there.

As shown in the drawing, the pocket 4 is open at its one end 5, but along its remaining three edges, is attached to the bandage 1 in a suitable fashion, for example by means of an elastic thread.

The upper, open end 5 of the pocket 4 is embodied as closable. To this end, a hook and loop fastener 6 is provided in the exemplary embodiment shown.

In the modified embodiment form of the holder according to FIG. 2, a wing-like flap 7 is provided. This can be thought of as a “pocket that is open on three sides” and, as shown, is attached to the bandage 1 along its lower edge 8 while its three other, open edges 9, 10, 11 can be fastened to the bandage 1. To this end, the bandage 1 has a corresponding, complementarily embodied fastening region 12 which, in cooperation with the flap edges 9, 10, 11, is likewise embodied as a hook and loop fastener in the exemplary embodiment shown.

Whereas the wing-like flap 7 in the embodiment form according to FIG. 2 is embodied as a rectangular flat piece, in the embodiment form according to FIG. 3, the flap 7′ is a triangular flat piece whose design otherwise corresponds to the one shown in FIG. 2.

As indicated by the arrows 13 and 13′, when the associated insert or fitting piece 3 is held pressed against the outside of the bandage 1, the respective wing-like flap 7 or 7′ is folded upward in the arrow direction and the insides of its edges 9, 10, 11 are brought into contact with the corresponding fastening region 12 or 12′ so that as a result, as with the pocket 4 of the embodiment form shown in FIG. 1, the insert or fitting piece 3 or 3′ is securely affixed to the bandage 1 in the desired position.

In the modified embodiment form according to FIG. 4, a hook and loop fastener area 14 is provided as a holder on the outside of the bandage and can be covered by a corresponding, complementarily embodied flat piece 15 for holding the fitting piece 3. As is shown in the drawing, this hook and loop fastener area 14 is embodied as a circumferential hook and loop fastener edge that cooperates with a correspondingly embodied hook and loop fastener edge provided on the inside of the flat piece.

Finally, as is clear from FIGS. 5 and 6, in this embodiment form, the holder is provided on the inside of the bandage 1 in the form of a hook and loop fastener area 17 that extends over the entire sole region. The fitting piece 3 can be detachably fastened to this hook and loop fastener area 17 in virtually any desired position. To this end, one side of the fitting piece, i.e. its “underside”, also has a hook and loop fastener surface 18 that is complementarily embodied in the usual fashion and cooperates with the hook and loop fastener surface 17 on the bandage to produce a detachable, yet secure and precise hold of the fitting piece 3.

With regard to remaining defining characteristics of the invention that have not been explained in detail, express reference is hereby made to the claims and the drawings.

1-11. (canceled)

12. An elastic bandage for holding an orthopedic or orthotic element adjacent an injured or injury-prone foot, comprising:
A) an elastic sleeve or tube sized and shaped to fit over a foot, the elastic sleeve or tube including a sole region positioned adjacent the sole of the foot when the bandage is fitted thereon; and
B) a holder in the form of a hook and loop fastener area provided on the inside of the sleeve or tube and extending over the entire sole region;
whereby an orthopedic or orthotic element provided with a complementary hook and loop fastening area can be detachably fastened to the holder at any desired position on the sole region.
